(19) United States
(12) Patent Application Publication
(10) Pub. No.: US 2017/0296465 A1
(43) Pub. Date: Oct. 19, 2017

(54) MICRONEEDLE ARRAY

(71) Applicant: FUJIFILM Corporation, Tokyo (JP)

(72) Inventors: Junya YOSHIDA, Kanagawa (JP); Ikuo KAWAUCHI, Kanagawa (JP); Yasushi MOROHASHI, Kanagawa (JP)

(73) Assignee: FUJIFILM Corporation, Tokyo (JP)

(21) Appl. No.: 15/486,312

(22) Filed: Apr. 13, 2017

(30) Foreign Application Priority Data
Apr. 15, 2016 (JP) 2016-081777

(51) Int. Cl. A61K 9/00; A61K 47/26; A61K 47/36; A61M 37/00; A61K 38/27; B29C 39/02; B29C 39/26; B29L 31/00; B29K 105/00

(52) U.S. Cl. CPC A61K 9/0021; B29C 39/026; A61K 47/26; A61K 47/36; A61M 37/0015; A61K 38/27; B29C 39/26; A61M 2037/0023; A61M 2037/0061; A61M 2037/0053; B29K 2105/0035; B29K 2105/0058; B29K 2883/00; B29L 2031/7544; B29L 2031/756

(57) ABSTRACT

To provide a microneedle array which includes a sheet and needles, improves transfer of a medicament into the blood, and is capable of achieving high drug efficacy.

Provided is a microneedle array including: a sheet; and a plurality of needles present on the upper surface of the sheet, in which the needles contain a water-soluble polymer and a medicament, the sheet contains a water-soluble polymer, and the administration is performed such that 20 μm≦L2≦L−L1 is satisfied, here, L represents the length of a needle, L1 represents the length of a needle tip region, which contains 90% of the total medicament in the microneedle array, from the needle tip, L2 represents the average remaining length of the needle after administration using the microneedle array, and the unit of L, L1, and L2 is μm.

### CROSS-REFERENCE TO RELATED APPLICATIONS

The present application claims priority under 35 U.S.C. §119 to Japanese Patent Application No. 2016-81777 filed on Apr. 15, 2016. The above application is hereby expressly incorporated by reference, in its entirety, into the present application.

### BACKGROUND OF THE INVENTION

### 1. Field of the Invention

The present invention relates to a microneedle array.

### 2. Description of the Related Art

As a method of administering a medicament to the surface of an organism such as the skin or the mucous membrane, a method of adhering a liquid substance or a powdery substance to the surface of an organism may be exemplified. Further, in biopharmaceuticals which have been attracting attention recently, since it is extremely difficult to enter a barrier layer through infiltration, a method of administering the pharmaceuticals through an injection is selected.

Further, a method of administering a medicament used to administer an appropriate amount of medicament and achieve sufficient drug efficacy, a method of injecting a medicament into the skin without pain by microneedles penetrating a horny barrier layer using a microneedle array in which microneedles (needles) which contain a medicament and have a high aspect ratio are formed has been attracting attention. For example, a self-dissolving microneedle array that uses a substance having solubility in vivo as a base material has been reported. In the self-dissolving microneedle array, a medicament can be intradermally administered by allowing the base material to hold a medicament and the base material being self-dissolved when microneedles are inserted into the skin.

WO2009/66763A discloses a needle-like formulation for a body surface which includes a base formed of a substance having solubility in vivo and a target substance held by the base and is used by being inserted into a body surface, and the target substance is absorbed into the body through dissolution of the base. Further, the formulation for a body surface is configured of two or more sections divided in an insertion direction, and the target substance is held in at least one section other than the section at the rearmost end. The section in which the target substance is held is obtained by solidifying the base that dissolves the target substance.

JP2016-30072A discloses a microneedle array which includes a sheet and a plurality of needles present on the upper surface of the sheet, in which the needles contain a water-soluble polymer and a medicament, and the sheet contains a water-soluble polymer and sugar alcohol.

### SUMMARY OF THE INVENTION

When the method of adhering a medicament which is a liquid substance or a powdery substance to the surface of an organism is used, since the region to which the medicament adheres is limited to the surface of the skin, the adhering medicament is occasionally removed due to perspiration or contact with foreign matter. Therefore, it is difficult to administer an appropriate amount of medicament. Further, according to such a method of using infiltration resulting from diffusion of the medicament, since the infiltration of the medicament is inhibited by a horny barrier layer, it is difficult to obtain sufficient drug efficacy. In addition, administration of the medicament through an injection is associated with pain or infection risk because the injection needs to be performed by medical workers.

As a replacement of the method of administering a medicament through an injection, a method of injecting a medicament into the skin using a microneedle array has been attracting attention, but the administration method using a microneedle array is difficult to obtain the same drug efficacy as the administration method through an injection. In WO2009/66763A and JP2016-30072A, there is no description about the relationship between the drug efficacy and the administration method of the time for administration.

An object of the present invention is to provide a microneedle array which includes a sheet and needles, improves transfer of a medicament into the blood, and is capable of achieving high drug efficacy.

The present inventors conducted intensive research in order to solve the above-described problems. As the result, it was found that most of the medicament in the microneedle array is transferred into the blood and the medicament administered through holes in the skin which are opened by the needles of the microneedle array can be administered without flowing out of the skin by administering the microneedle array such that a region containing a medicament in needles of the microneedle array is completely dissolved and a root region which does not contain a medicament in needles or has a small content of the medicament is not dissolved, and thus the same drug efficacy as in the case of subcutaneous injection can be obtained. The present invention has been completed based on these findings.

In other words, according to the present invention, the following inventions are provided.

[1] A microneedle array comprising: a sheet; and a plurality of needles present on an upper surface of the sheet, in which the needles contain a water-soluble polymer and a medicament, the sheet contains a water-soluble polymer, and administration is performed such that 20 μm≦L2≦L−L1 is satisfied, where, L represents a length of a needle, L1 represents a length of a needle tip region, which contains 90% of the total medicament in the microneedle array, from a needle tip, L2 represents an average remaining length of needles after the administration using the microneedle array, and a unit of L, L1, and L2 is μm.

[2] The microneedle array according to [1], in which the administration is performed such that 20 μm≦L2=L−L1 is satisfied.

[3] The microneedle array according to [1] or [2], further comprising: a plurality of frustums which are present between the sheet and the plurality of needles, the needle tip region contains a first water-soluble polymer and a medicament, and a root region other than the needle tip region, a frustum, and the sheet of the needle contain a second water-soluble polymer.

[4] The microneedle array according to any one of [1] to [3], in which the needle tip region further contains disaccharides.

[5] The microneedle array according to [4], in which the disaccharides are one or more selected from sucrose, maltose, and trehalose.

[6] The microneedle array according to any one of [1] to [5], in which the water-soluble polymers of a needle and a sheet each independently are at least one selected from the group consisting of hydroxyethyl starch, dextran, chondroitin sulfate, sodium chondroitin sulfate, sodium hyaluronate, carboxymethyl cellulose, polyvinylpyrrolidone, polyoxyethylene polyoxypropylene glycol, polyethylene glycol, and polyvinyl alcohol.

[7] The microneedle array according to any one of [1] to [6], in which the medicament is a peptide hormone.

[8] A method of administering a microneedle array comprising: administering a microneedle array which includes a sheet; and a plurality of needles present on an upper surface of the sheet and in which the needles contain a water-soluble polymer and a medicament, and the sheet contains a water-soluble polymer, to a subject such that 20 μm≦L2≦L−L1 is satisfied, where, L represents a length of a needle, L1 represents a length of a needle tip region, which contains 90% of the total medicament in the microneedle array, from a needle tip, L2 represents an average remaining length of needles after administration using the microneedle array, and a unit of L, L1, and L2 is μm.

[9] The method of administering a microneedle array according to [8], in which the administration is performed such that 20 μm≦L2=L−L1 is satisfied.

[10] The method of administering a microneedle array according to [8] or [9], in which the microneedle array includes a plurality of frustums between the sheet and the plurality of needles, the needle tip region contains a first water-soluble polymer and a medicament, and a root region other than the needle tip region, a frustum, and the sheet of the needle contain a second water-soluble polymer.

[11] The method of administering a microneedle array according to any one of [8] to [10], in which the needle tip region further contains disaccharides.

[12] The method of administering a microneedle array according to [11], in which the disaccharides are one or more selected from sucrose, maltose, and trehalose.

[13] The method of administering a microneedle array according to any one of [8] to [12], in which the water-soluble polymers of a needle and a sheet each independently are at least one selected from the group consisting of hydroxyethyl starch, dextran, chondroitin sulfate, sodium chondroitin sulfate, sodium hyaluronate, carboxymethyl cellulose, polyvinylpyrrolidone, polyoxyethylene polyoxypropylene glycol, polyethylene glycol, and polyvinyl alcohol.

[14] The method of administering a microneedle array according to any one of [8] to [13], in which the medicament is a peptide hormone.

According to the present invention, it is possible to provide a microneedle array which includes a sheet and a plurality of needles present on the sheet and is capable of achieving high drug efficacy.

### BRIEF DESCRIPTION OF THE DRAWINGS

FIG. 1 is a view illustrating a length L of a needle and a length L1 of a needle tip region, which contains 90% of the total medicament in the microneedle array, from the needle tip.

FIG. 2A is a perspective view illustrating a conical microneedle, FIG. 2B is a perspective view illustrating a pyramid-like microneedle, and FIG. 2C is a cross-sectional view illustrating a conical and pyramid-like microneedle.

FIG. 3 is a perspective view illustrating a microneedle in another shape.

FIG. 4 is a perspective view illustrating a microneedle in another shape.

FIG. 5 is a cross-sectional view of the microneedles illustrated in FIGS. 3 and 4.

FIG. 6 is a perspective view illustrating a microneedle in another shape.

FIG. 7 is a perspective view illustrating a microneedle in another shape.

FIG. 8 is a cross-sectional view of the microneedles illustrated in FIGS. 6 and 7.

FIG. 9 is a cross-sectional view of a microneedle in another shape in which the inclination (angle) of the side surface of the needle is continuously changed.

FIGS. 10A to 10C are process views illustrating a method of producing a mold.

FIG. 11 is an enlarged view of a mold.

FIG. 12 is a cross-sectional view illustrating a mold in another shape.

FIGS. 13A to 13C are views schematically illustrating a process of filling the mold with a polymer-dissolved solution containing a medicament.

FIG. 14 is a perspective view illustrating the tip of the nozzle.

FIG. 15 is a partially enlarged view of the tip of the nozzle and the mold during filling.

FIG. 16 is a partially enlarged view of the tip of the nozzle and the mold during transfer.

FIGS. 17A to 17D are views describing a process of forming another microneedle array.

FIGS. 18A to 18C are views describing a process of forming another microneedle array.

FIG. 19 is a view describing a peeling process.

FIG. 20 is a view describing another peeling process.

FIG. 21 is a view describing a microneedle array.

FIGS. 22A and 22B are respectively a plan view and a side view of an original plate.

FIG. 23 is a view schematically illustrating a filling device used in examples.

FIGS. 24A to 24E are views illustrating mechanisms of the present invention and comparative examples.

### DESCRIPTION OF THE PREFERRED EMBODIMENTS

Hereinafter, embodiments of the present invention will be described in detail.

In the present specification, the expression “containing a medicament” means that a medicament having an amount enough to exhibit drug efficacy is contained when the body surface is punctured. The expression “not containing a medicament” means that a medicament having an amount enough to exhibit drug efficacy is not contained, and the range of the amount of the medicament covers from a case where the medicament is not contained at all to a case where the amount thereof is not enough to exhibit the drug efficacy.

[Configuration of Microneedle Array]

A microneedle array of the present invention includes a sheet and a plurality of needles present on the upper surface of the sheet, in which the needles contain a water-soluble polymer and a medicament, the sheet contains a water-soluble polymer, and the administration is performed such that 20 μm≦L2≦L−L1 is satisfied. In the present invention, it is preferable that the administration is performed such that 20 μm≦L2=L−L1 is satisfied. Here, L represents the length of a needle, L1 represents the length of a needle tip region, which contains 90% of the total medicament in the microneedle array, from the needle tip, L2 represents the average remaining length of needles after administration using the microneedle array, and the unit of L, L1, and L2 is μm. The administration can be performed such that 20 μm≦L2≦L−L1 is satisfied by adjusting the time for administration according to the type of the water-soluble polymer constituting the microneedle array.

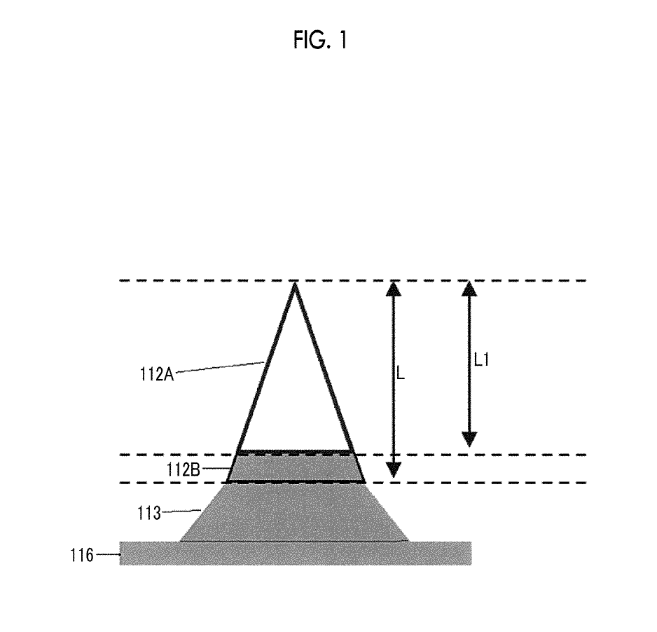

The microneedle array illustrated in FIG. 1 includes a sheet 116, a frustum 113, a needle tip region 112A, and a needle root region 112B. In FIG. 1, L represents the length of the needle and L1 represents the length of the needle tip region 112A, which contains 90% of the total medicament in the microneedle array, from the needle tip. The region corresponding to L1 in the needle is referred to as the needle tip region and the region corresponding a region other than L1 in the needle is referred to as the needle root region.

L2 represents an average remaining length of needles after administration of the microneedle array. The average remaining length of needles after administration indicates the average length of needles in the microneedle array which is administered and peeled from the skin.

L, L1, and L2 can be measured using a stereoscopic microscope. The average remaining length represented by L2 is the average remaining length of total needles.

In a case of administering a medicament using the microneedle array, it is difficult to obtain the same drug efficacy as an injection. The present inventors found that the reason for this is that the medicament which is administered through-holes opened by the needles of the microneedle array flows out because the microneedle array is continuously fixed into the skin after the microneedle array is punctured into the skin and the needles of the microneedle array are dissolved. FIG. 24A illustrates a state of the microneedle array immediately after being punctured into the skin. The skin is configured of hypodermis 71 and epidermis 72. FIG. 24B illustrates a state in which the needle tip region containing the medicament in a needle is dissolved in the skin after the microneedle array is punctured. FIGS. 24C and 24D illustrate a case where the microneedle array is fixed into the skin after needles are dissolved. FIG. 24C illustrates a state in which a hole is opened by the dissolution of the entire needle so that the medicament and the body fluid ooz out from the hole. FIG. 24D illustrates a medicament 73 which is leaked out from the hole opened in the skin. FIG. 24E illustrates a case where the microneedle array is peeled off before needles are dissolved. In FIG. 24E, the body fluid and the medicament are not leaked out by blocking the hole in the skin before the body fluid and the medicament ooz out.

In the present invention, the same drug efficacy as that of an injection can be obtained using the microneedle array by performing administration such that 20 μm≦L2≦L−L1 is satisfied, based on the above-described findings. The achievement of high drug efficacy by employing the above-described configuration of present invention is an effect that cannot be expected at all from the past.

In the present invention, plural means one or more.

The microneedle array of the present invention includes at least a sheet or needles and a medicament is carried in the needles in order to efficiently administer the medicament into the skin.

The microneedle array of the present invention is a device in which a plurality of needles are arranged in an array on the upper surface side of the sheet. It is preferable that the needles are arranged on the upper surface side of the sheet. The needles may be directly arranged on the upper surface of the sheet or may be arranged on the upper surfaces of frustums disposed on the upper surface of the sheet.

It is preferable that the needles are arranged on the upper surfaces of frustums arranged on the upper surface of the sheet. In this case, the microneedle array of the present invention includes a plurality of frustums between the sheet and the plurality of needles. According to this aspect, it is preferable that the needle tip region contains a first water-soluble polymer and a medicament, and the root region other than the needle tip region in a needle, a frustum, and the sheet contain a second water-soluble polymer. The first water-soluble polymer and the second water-soluble polymer may be the same as or different from each other. The water-soluble polymer will be described later.

The sheet is a foundation for supporting needles and has a planar shape as the shape of the sheet 116 illustrated in FIGS. 1 to 9. At this time, the upper surface of the sheet indicates the surface on which the plurality of needles are arranged in an array.

The area of the sheet is not particularly limited, but is preferably in a range of 0.005 to 1000 mm², more preferably in a range of 0.05 to 500 mm², and still more preferably in a range of 0.1 to 400 mm².

The thickness of the sheet is a distance between the surface in contact with frustums or needles and the surface on the opposite side. The thickness of the sheet is preferably in a range of 1 μm to 2000 μm, more preferably in a range of 3 μm to 1500 μm, and still more preferably in a range of 5 μm to 1000 μm.

The sheet contains a water-soluble polymer. The sheet may be formed of a water-soluble polymer or may contain other additives (for example, disaccharides). Further, it is preferable that the sheet does not contain a medicament.

The water-soluble polymer contained in the sheet is not particularly limited, and examples thereof include polysaccharides, polyvinylpyrrolidone, polyoxyethylene polyoxypropylene glycol, polyethylene glycol, polyvinyl alcohol, and protein (for example, gelatin). Examples of the polysaccharides include hyaluronic acid, sodium hyaluronate, pullulan, dextran, dextrin, chondroitin sulfate, sodium chondroitin sulfate, a cellulose derivative (for example, a water-soluble cellulose derivative obtained by partially modifying cellulose such as carboxymethyl cellulose, hydroxypropyl cellulose, or hydroxypropyl methylcellulose), hydroxyethyl starch, and gum Arabic. The above-described components may be used alone or in combination of two or more kinds thereof.

Among these, as the water-soluble polymer contained in the sheet, at least one selected from the group consisting of hydroxyethyl starch, dextran, chondroitin sulfate, sodium chondroitin sulfate, sodium hyaluronate, carboxymethyl cellulose, polyvinylpyrrolidone, polyoxyethylene polyoxypropylene glycol, polyethylene glycol, and polyvinyl alcohol is preferable and chondroitin sulfate is particularly preferable.

Disaccharides may be added to the sheet and examples of the disaccharides include sucrose, lactulose, lactose, maltose, trehalose, and cellobiose. Among these, sucrose, maltose, and trehalose are particularly preferable.

The microneedle array is configured of a plurality of needles arranged in an array on the upper surface of the sheet. The needles have a projected structure with a tip, and the shape thereof is not limited to a needle shape having a sharp tip and may be a shape with a blunt tip.

Examples of the shape of a needle include a conical shape, a polygonal pyramid shape (square pyramid shape or the like), and a spindle shape. For example, a needle may have a shape of the needle 112 illustrated in any of FIGS. 1 to 9, in which the entire shape of the needle may be a conical shape, a polygonal pyramid shape (square pyramid shape or the like), or a shape of a structure in which the inclination (angle) of the side surface of the needle is continuously changed. Further, a needle may have a multilayer structure with two or more layers, in which the inclination (angle) of the side surface of the needle is discontinuously changed.

In a case where the microneedle array of the present invention is applied to the skin, it is preferable that the needles are inserted into the skin and the upper surface or a part of the sheet is brought into contact with the skin.

The height (length) of a needle indicates the length of a perpendicular line drawn from the tip of the needle to a frustum or the sheet (in a case where a frustum is not present). The height (length) of a needle is not particularly limited, but is preferably in a range of 50 μm to 3000 μm, more preferably in a range of 100 μm to 1500 μm, and still more preferably in a range of 100 μm to 1000 μm. It is preferable that the length of a needle is 50 μm or greater because a medicament can be percutaneously administered. Further, it is preferable that the length of a needle is 3000 μm or less because occurrence of pain resulting from the contact of needles with the nerve is prevented and bleeding can be avoided.

The interface between a frustum (or a needle in a case where a frustum is not present) and the sheet is referred to as a base. The distance between a base of one needle and a point farthest from the base is preferably in a range of 50 μm to 2000 μm, more preferably in a range of 100 μm to 1500 μm, and still more preferably in a range of 200 μm to 1000 μm.

The number of needles to be arranged in one microneedle array is preferably in a range of 1 to 2000, more preferably in a range of 3 to 1000, and still more preferably in a range of 5 to 500. In a case where one microneedle array includes two needles, the interval between needles indicates the distance between feet of each perpendicular line drawn from the tip of a needle to a frustum or the sheet (in the case where a frustum is not present). In a case where one microneedle array includes three or more needles, the interval between needles to be arranged indicates an average value obtained by acquiring the distance between a foot of a perpendicular line drawn from the tip of a needle to a frustum or the sheet (in the case where frustums are not present) and a foot of a perpendicular line drawn from the tip of a needle closest to the needle to a frustum or the sheet and averaging the values obtained from all needles. The interval between needles is preferably in a range of 0.1 mm to 10 mm, more preferably in a range of 0.2 mm to 5 mm, and still more preferably in a range of 0.3 mm to 3 mm.

The needles contain a water-soluble polymer and a medicament.

It is preferable that the water-soluble polymer is a biosoluble substance such that a human body is not damaged even when needles remain in the skin.

The water-soluble polymer contained in the needles is not particularly limited, and examples thereof include polysaccharides, polyvinylpyrrolidone, polyoxyethylene polyoxypropylene glycol, polyethylene glycol, polyvinyl alcohol, and protein (for example, gelatin). Examples of the polysaccharides include hyaluronic acid, sodium hyaluronate, pullulan, dextran, dextrin, chondroitin sulfate, sodium chondroitin sulfate, a cellulose derivative (for example, a water-soluble cellulose derivative obtained by partially modifying cellulose such as carboxymethyl cellulose, hydroxypropyl cellulose, or hydroxypropyl methylcellulose), hydroxyethyl starch, and gum Arabic. The above-described components may be used alone or in combination of two or more kinds thereof.

Among these, as the water-soluble polymer contained in the needles, at least one selected from the group consisting of hydroxyethyl starch, dextran, chondroitin sulfate, sodium chondroitin sulfate, sodium hyaluronate, carboxymethyl cellulose, polyvinylpyrrolidone, polyoxyethylene polyoxypropylene glycol, polyethylene glycol, and polyvinyl alcohol is preferable and hydroxyethyl starch is particularly preferable. Further, polysaccharides typically with no charge are more preferable because polysaccharides are unlikely to be aggregated when mixed with a medicament. The water-soluble polymer contained in the needles may be the same as or different from the water-soluble polymer contained in the sheet.

Disaccharides may be added to the needles (particularly, the needle tip region) and examples of the disaccharides include sucrose, lactulose, lactose, maltose, trehalose, and cellobiose. Among these, sucrose, maltose, and trehalose are preferable.

In the present invention, the content of the water-soluble polymer is 50% by mass or greater, preferably 55% by mass or greater, more preferably 60% by mass or greater, and still more preferably 65% by mass or greater with respect to the total solid content of the needles.

The upper limit thereof is not particularly limited, but the content of the water-soluble polymer is preferably 99% by mass or less, more preferably 95% by mass or less, and still more preferably 90% by mass or less with respect to the total solid content of the needles.

When the content of the water-soluble polymer is set to be 50% by mass or greater with respect to the total solid content of the needles, excellent puncture properties and excellent drug efficacy can be obtained.

The proportion of the water-soluble polymer in the total solid content of the needles can be measured using the following method, but the method of measuring the proportion thereof is not particularly limited. As an example of the measurement method, needles of a prepared microneedle array are cut, the needles are dissolve in a buffer solution (a buffer solution suitable for dissolving the water-soluble polymer constituting the needles, such as phosphate buffered saline (PBS) or the like), and then the amount of the water-soluble polymer in the solution can be measured according to a high performance liquid chromatography method.

The needles contain a medicament.

The medicament indicates a substance that affects a human body. It is preferable that the medicament is selected from peptides (including peptide hormones or the like) or derivatives thereof, protein, a nucleic acid, polysaccharides, vaccine, adjuvant, a pharmaceutical compound belonging to a water-soluble low molecular compound, or cosmetic ingredients. The molecular weight of the medicament is not particularly limited, but a medicament having a molecular weight of 500 or greater is preferable in a case of protein.

Examples of peptides or derivatives thereof and protein include calcitonin, adrenocorticotropic hormone, parathyroid hormone (PTH), human PTH (1→34), insulin, exendin, secretin, oxytocin, angiotensin, β-endorphin, glucagon, vasopressin, somatostatin, gastrin, luteinizing hormone releasing hormone, enkephalin, neurotensin, atrial natriuretic peptide, growth hormone, growth hormone releasing hormone, bradykinin, substance P, dynorphin, thyroid stimulating hormone, prolactin, interferon, interleukin, granulocyte colony stimulating factor (G-CSF), glutathione peroxidase, superoxide dismutase, desmopressin, somatomedin, endothelin, and salts of these.

Examples of the vaccine include influenza antigen (influenza vaccine), hepatitis B virus surface antigen (HBs) antigen, hepatitis Be antigen (HBe antigen), Bacille de calmette et Gaerin (BCG) antigen, measles antigen, rubella antigen, varicella antigen, yellow fever antigen, shingles antigen, rotavirus antigen, influenza bacilli b type (Hib) antigen, rabies antigen, cholera antigen, diphtheria antigen, pertussis antigen, tetanus antigen, inactivated polio antigen, Japanese encephalitis antigen, human papilloma antigen, and antigens obtained by mixing two to four types of these.

Examples of the adjuvant include aluminum salts such as aluminum phosphate, aluminum chloride, and aluminum hydroxide, emulsions such as MF59 (registered trademark) and AS03 (trade name), liposomes, plant-derived components, a nucleic acid, biopolymers, cytokine, peptides, protein, sugar chain.

Among these, as the medicament, at least one selected from the group consisting of peptide hormones, vaccines, and adjuvants is preferable, peptide hormones are particularly preferable. Among peptide hormones, growth hormone is particularly preferable.

The content of the medicament in all needles is not particularly limited, but is preferably in a range of 1 to 60% by mass, more preferably in a range of 1 to 50% by mass, and particularly preferably in a range of 1 to 45% by mass with respect to the mass of the solid content of needles.

Hereinafter, preferred embodiments of the present invention will be described with reference to the accompanying drawings, but the present invention is not limited thereto.

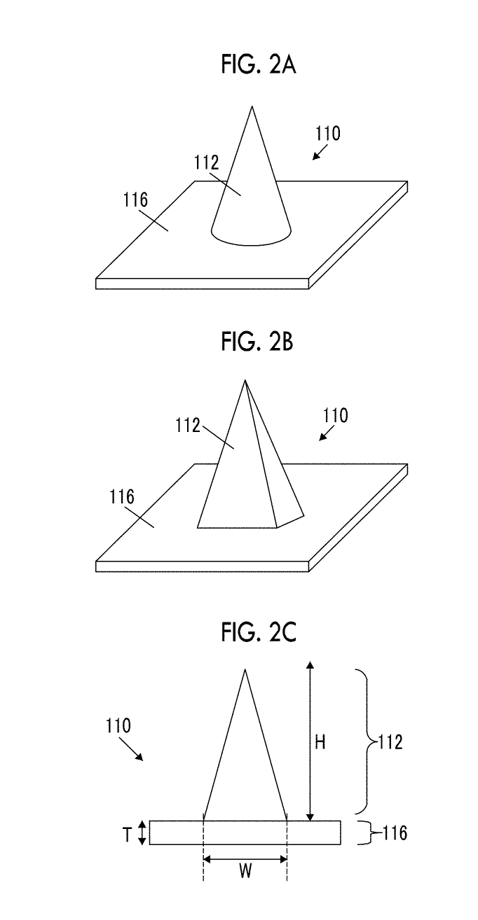

FIGS. 2 to 9 are partially enlarged views illustrating a microneedle 110 in the microneedle array. The microneedle array of the present invention is configured by the plurality of needles 112 being formed on the surface of the sheet 116 (in the figured, one needle 112 is shown on the sheet 116 or one frustum 113 and one needle 112 are shown on the sheet 116 and this is referred to as the microneedle 110).

The needle 112 has a conical shape in FIG. 2A and the needle 112 has a square pyramid shape in FIG. 2B. In FIG. 2C, H represents the height of the needle 112, W represents the diameter (width) of the needle 112, and T represents the height (thickness) of the sheet 116.

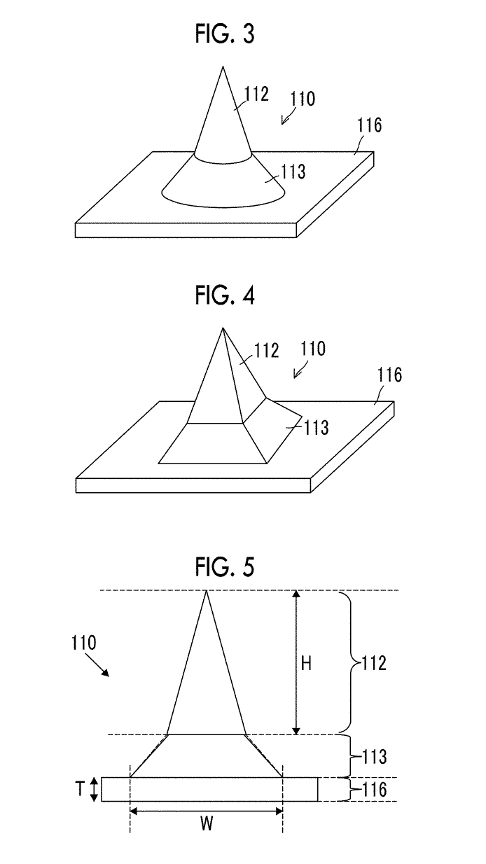

FIGS. 3 and 4 illustrate microneedles 110, on which the frustum 113 and the needle 112 are formed and which have different shapes, formed on the surface of the sheet 116. In FIG. 3, the frustum 113 has a truncated conical shape and the needle 112 has a conical shape. In FIG. 4, the frustum 113 has a truncated square pyramid shape and the needle 112 has a square pyramid shape. However, the shape of the needle is not particularly limited.

FIG. 5 is a cross-sectional view illustrating the microneedles 110 illustrated in FIGS. 3 and 4. In FIG. 5, H represents the height of the needle 112, W represents the diameter (width) of the base, and T represents the height (thickness) of the sheet 116.

It is preferable that the microneedle array of the present invention has a shape of the microneedle 110 of FIG. 5 other than the shape of the microneedle 110 in FIG. 2C. With such a configuration, the volume of all needles becomes larger so that a greater amount of medicament can be concentrated on the tip of a needle when the microneedle array is produced.

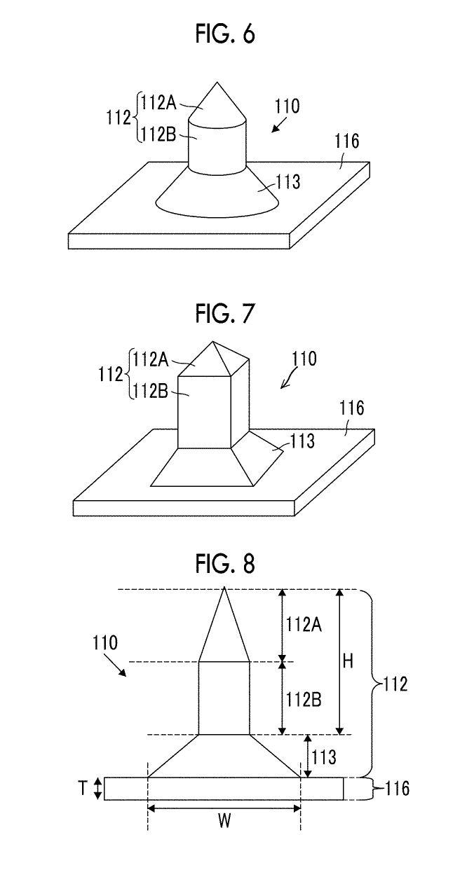

FIGS. 6 and 7 illustrate microneedles 110 in different shapes.

A first layer 112A of the needle illustrated in FIG. 6 has a conical shape and a second layer 112B of the needle in FIG. 6 has a columnar shape. The first layer 112A of the needle illustrated in FIG. 7 has a square pyramid shape and the second layer 112B of the needle in FIG. 7 has a square columnar shape. However, the shape of a needle is not limited to these shapes.

FIG. 8 is a cross-sectional view illustrating the microneedles 110 illustrated in FIGS. 6 and 7. In FIG. 8, H represents the height of the needle 112, W represents the diameter (width) of the base, and T represents the height (thickness) of the sheet 116.

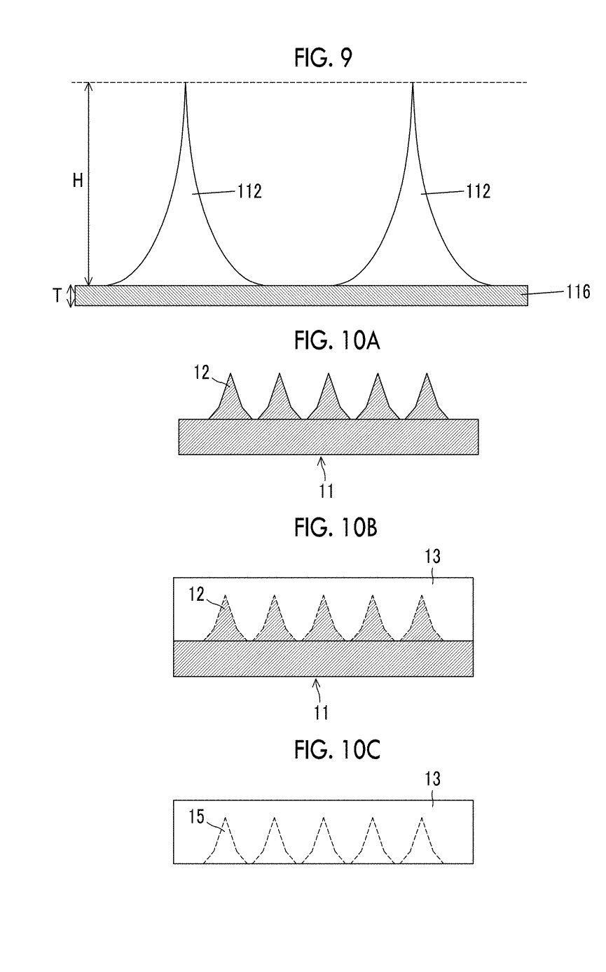

FIG. 9 is a cross-sectional view of a microneedle in another shape in which the inclination (angle) of the side surface of the needle 112 is continuously changed. In FIG. 9, H represents the height of the needle 112 and T represents the height (thickness) of the sheet 116.

In the microneedle array of the present invention, it is preferable that needles are arranged at intervals of approximately 0.1 to 10 needles per 1 mm in a row. It is more preferable that the microneedle array has 1 to 10000 microneedles per 1 cm². When the density of microneedles is set to 1 needle/cm² or greater, the microneedles can efficiently puncture the skin. When the density of the microneedles is set to 10000 needles/cm² or less, the microneedle array can sufficiently puncture the skin. The density of needles is preferably in a range of 10 to 5000 needles/cm², more preferably in a range of 25 to 1000 needles/cm², and particularly preferably in a range of 25 to 400 needles/cm².

The microneedle array of the present invention can be supplied in a sealed storage form together with a drying agent. As the drying agent, known drying agents (such as silica gel, calcined lime, calcium chloride, silica alumina, and a sheet-like drying agent) can be used.

[Method of Producing Microneedle Array]

The microneedle array of the present invention can be produced by the following method in conformity with the method described in, for example, JP2013-153866A or WO2014/077242A.

(Preparation of Mold)

FIGS. 10A to 10C are process views illustrating a method of preparing a mold (die). As illustrated in FIG. 10A, first, an original plate is prepared used to prepare the mold. There are two methods for preparing an original plate 11.

According to the first method, a Si substrate is coated with a photoresist, exposed, and then developed. Further, an array of shaped portions 12 having a conical shape (projection) is prepared on the surface of the original plate 11 by performing etching using reactive ion etching (RIE) or the like. In addition, when the etching such as RIE or the like is performed so as to form shaped portions having a conical shape on the surface of the original plate 11, the portions having a conical shape can be formed by performing etching in an oblique direction while the Si substrate rotate. According to the second method, an array of the shaped portions 12 having a square pyramid shape or the like is formed on the surface of the original plate 11 by performing processing on a metal substrate such as Ni using a cutting tool such as a diamond bit.

Next, a mold is prepared. Specifically, a mold 13 is prepared using the original plate 11 as illustrated in FIG. 10B. As the method of preparing the mold, four methods are considered.

According to the first method, a silicone resin obtained by adding a curing agent to polydimethylsiloxane (PDMS, for example, SYLGARD 184 (registered trade mark, manufactured by Dow Corning Toray Co., Ltd.)) is poured into the original plate 11, subjected to a heat treatment at 100° C., cured, and peeled from the original plate 11. According to the second method, an ultraviolet (UV) cured resin which is cured by being irradiated with ultraviolet rays is poured into the original plate 11, irradiated with ultraviolet rays in a nitrogen atmosphere, and peeled off from the original plate 11. According to the third method, a solution obtained by dissolving a plastic resin such as polystyrene or polymethyl methacrylate (PMMA) in an organic solvent is poured into the original plate 11 coated with a peeling agent, dried so that the organic solvent is volatilized, and cured, and then peeled off from the original plate 11. According to the fourth method, an inverted product is produced using Ni electroforming.

In this manner, the mold 13 formed by needle-like recesses 15, which have an inverted shape of the conical shape or the pyramid shape of the original plate 11, being two-dimensionally arranged is prepared. The mold 13 prepared in the above-described manner is illustrated in FIG. 10C.

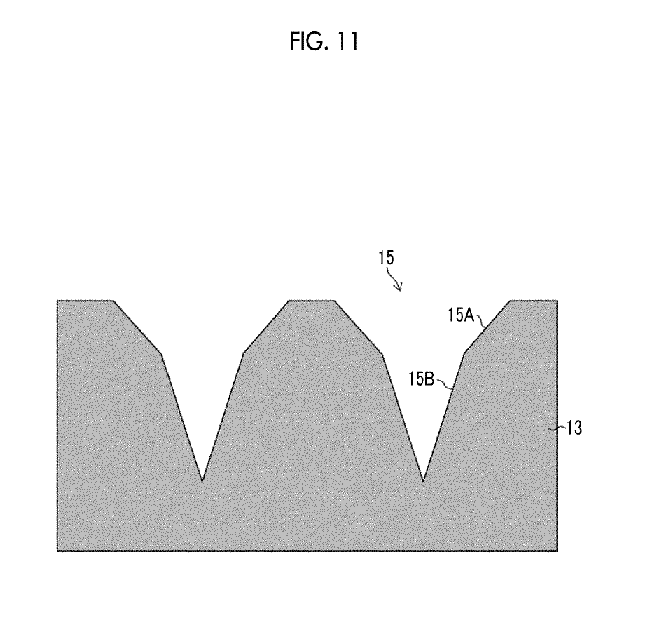

FIG. 11 illustrates another preferred embodiment of the mold 13. The needle-like recess 15 includes a tapered inlet portion 15A which becomes narrower in a depth direction from the surface of the mold 13 and a tip recess 15B which becomes tapered in the depth direction. When the inlet portion 15A has a tapered shape, the needle-like recess 15 is easily filled with the water-soluble polymer-dissolved solution.

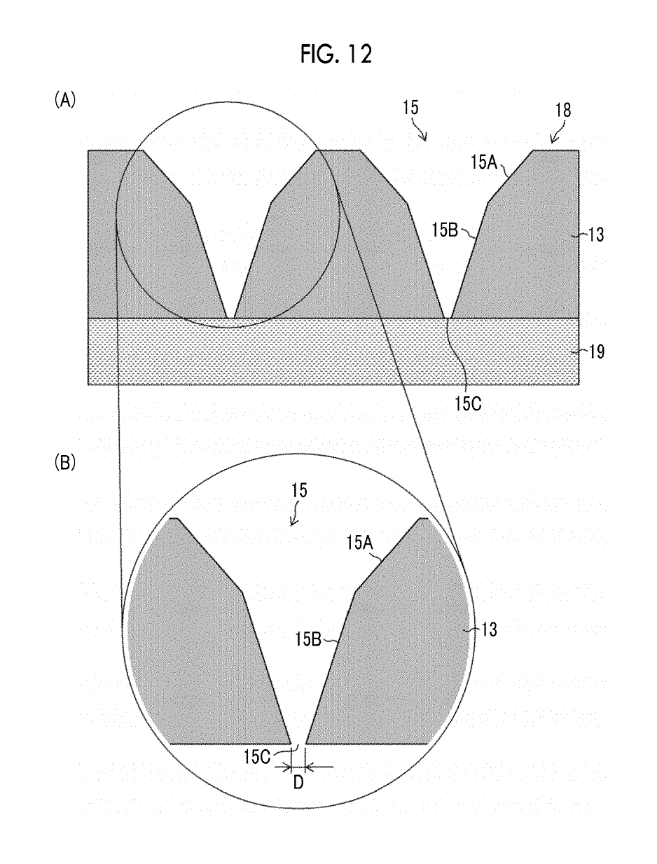

FIG. 12 illustrates a more preferred embodiment of a mold complex 18 at the time of producing the microneedle array. The (A) portion of FIG. 12 illustrates the mold complex 18. The (B) portion of FIG. 12 is a partially enlarged view of a portion enclosed by a circle in the (A) portion.

As illustrated in the (A) portion of FIG. 12, the mold complex 18 includes the mold 13 having an air vent hole 15C formed on the tip (bottom) of the needle-like recess 15; and an air permeating sheet 19 which is bonded to the rear surface of the mold 13 and is formed of a material that permeates a gas and does not permeate a liquid. The air vent hole 15C is formed as a through-hole penetrating the rear surface of the mold 13. Here, the rear surface of the mold 13 indicates the surface on a side on which the air vent hole 15C is formed. With this configuration, the tip of the needle-like recess 15 communicates with the air through the air vent hole 15C and the air permeating sheet 19.

When such a mold complex 18 is used, only the air present in the needle-like recess 15 can be released from the needle-like recess 15 without permeation of the polymer-dissolved solution filling the needle-like recess 15. In this manner, the property of transferring the shape of the needle-like recess 15 to a polymer becomes excellent in the above-described manner and a sharper needle can be formed.

A diameter D (diameter) of the air vent hole 15C is preferably in a range of 1 to 50 μm. In a case where the diameter D of the air vent hole 15C is less than 1 μm, the air vent hole 15C cannot be sufficiently used as an air bend hole. Further, in a case where the diameter D of the air vent hole 15C is greater than 50 μm, the sharpness of the tip of a formed microneedle is damaged.

As the air permeating sheet 19 formed of a material that permeates a gas and does not permeate a liquid, for example, an air permeating film (Poreflon (registered trade mart), FP-010, manufactured by Sumitomo Electric Industries, Ltd.) can be suitably used.

As the material used for the mold 13, an elastic material or a metal material can be used. Among these, an elastic material is preferable and a material having a high gas permeability is more preferable. The oxygen permeability, which is a representative example of the gas permeability, is preferably 1×10⁻¹² (mL/s·m2·Pa) or greater and more preferably 1×10⁻¹⁰ (mL/s·m²·Pa) or greater. Further, 1 mL is 10⁻⁶ m³. When the gas permeability is in the above-described range, the air present in a recess of the mold 13 can be released from the die and a microneedle array with less defects can be produced. Specific examples of such materials include materials obtained by melting or dissolving, in a solvent, a silicone resin (for example, SYLGARD 184 (registered trade mark, manufactured by Dow Corning Toray Co., Ltd.) or KE-1310ST (product number, manufactured by Shin-Etsu chemical Co., Ltd.)), a UV curable resin, or a plastic resin (for example, polystyrene or polyrnethyl methacrylate (PMMA)). Among these, a silicone rubber-based material is preferable since the material has durability to transfer resulting from repetitive pressure and has excellent peeling properties with respect to a material. Further, examples of the metal material include Ni, Cu, Cr, Mo, W, Ir, Tr, Fe, Co, MgO, Ti, Zr, Hf, V, Nb, Ta, α-aluminum oxide, zirconium oxide, stainless (for example, STAVAX (registered trademark) of Bohler-Uddeholm KK), and alloys thereof. As the material of a frame 14, the same material as the material of the mold 13 can be used.

(Water-Soluble Polymer-Dissolved Solution)

In the present invention, it is preferable to prepare a water-soluble polymer-dissolved solution containing a medicament used to form the needle tip region which is a part of a needle and a water-soluble polymer-dissolved solution used to form the needle root region and the sheet (or the needle root region, a frustum, and the sheet other than the needle tip region of a needle) other than the needle tip region of a needle.

The type of water-soluble polymer is as described in the present specification above.

Disaccharides may be mixed with both of the water-soluble polymer-dissolved solutions, and the type of disaccharides is as described in the present specification above.

The concentration of the water-soluble polymer in any of the water-soluble polymer-dissolved solutions varies depending on the type of the water-soluble polymer to be used, and is preferably in a range of 1 to 50% by mass. Further, a solvent used for dissolution may be a solvent other than hot water as long as the solvent has volatility, and methyl ethyl ketone (MEK) or alcohol can be used as the solvent.

(Formation of Needle Tip Region)

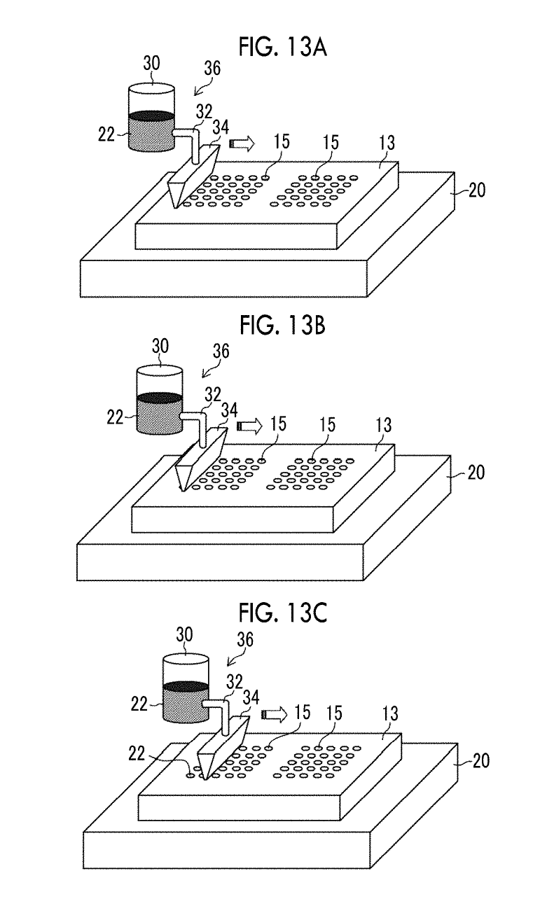

As illustrated in FIG. 13A, the mold 13 having needle-like recesses 15 which are two-dimensionally arranged is disposed on a base 20. In the mold 13, two sets of plural needle-like recesses 15 are formed such that 5 rows of needle-like recesses 15 and 5 columns of needle-like recesses 15 are two-dimensionally arranged. A liquid supply device 36 including a tank 30 which accommodates a water-soluble polymer-dissolved solution 22 containing a medicament; a pipe 32 which is connected with the tank; and a nozzle 34 which is connected with the tip of the pipe 32 is prepared. Further, in the present example, the case where 5 rows of needle-like recesses 15 and 5 columns of needle-like recesses 15 are two-dimensionally arranged is exemplified, but the number of the needle-like recesses 15 is not limited to 5 rows×5 columns as long as the needle-like recesses are two-dimensionally arranged in a manner of M×N (M and N each independently represent an arbitrary integer of 1 or greater, preferably in a range of 2 to 30, more preferably in a range of 3 to 25, and still more preferably in a range of 3 to 20).

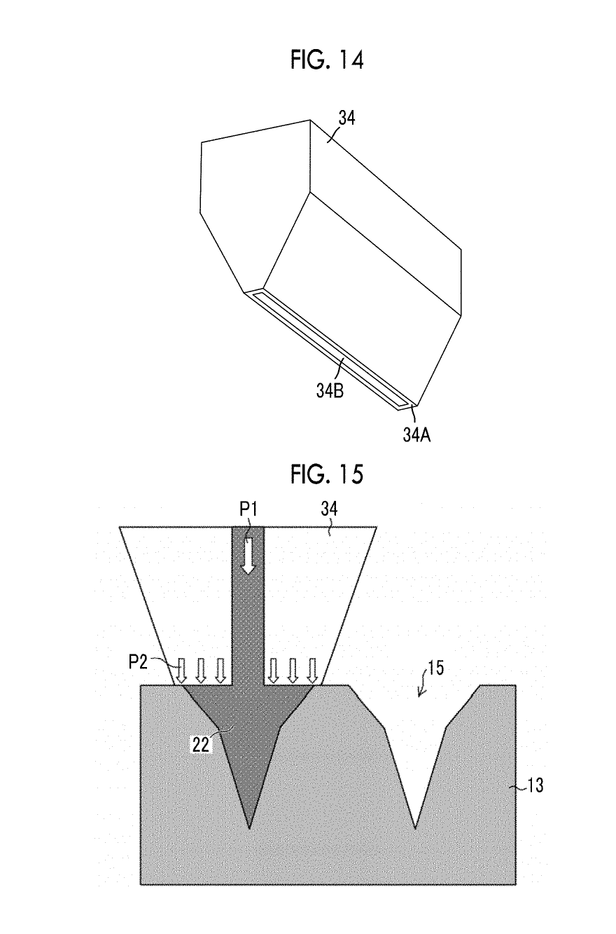

FIG. 14 is a perspective view schematically illustrating the tip portion of the nozzle. As illustrated in FIG. 14, the tip of the nozzle 34 includes a lip portion 34A which is a flat surface and an opening portion 34B having a slit shape. For example, a plurality of needle-like recesses 15 forming one row can be concurrently filled with the water-soluble polymer-dissolved solution 22 containing a medicament because of the opening portion 34B having a slit shape. The size (the length and the width) of the opening portion 34B can be suitably selected according to the number of needle-like recesses 15 to be filled with the water-soluble polymer-dissolved solution at the same time. When the length of the opening portion 34B is set to be large, a larger amount of needle-like recesses 15 can be filled with the water-soluble polymer-dissolved solution 22 containing a medicament at the same time. In this manner, the productivity can be improved.

As the material used for the nozzle 34, an elastic material or a metal material can be used. Examples thereof include TEFLON (registered trademark), stainless steel (steel special use stainless (SUS)), and titanium.

As illustrated in FIG. 13B, the position of the opening portion 34B of the nozzle 34 is adjusted on the needle-like recesses 15. The lip portion 34A of the nozzle 34 is in contact with the surface of the mold 13. The water-soluble polymer-dissolved solution 22 containing a medicament is supplied to the mold 13 from a liquid supply device 36, and the water-soluble polymer-dissolved solution 22 containing a medicament fills the needle-like recesses 15 from the opening portion 34B of the nozzle 34. In the present embodiment, a plurality of needle-like recesses 15 forming one row can be concurrently filled with the water-soluble polymer-dissolved solution 22 containing a medicament. However, the present invention is not limited thereto, and the needle-like recesses 15 can be filled with the water-soluble polymer-dissolved solution one by one.

In a case where the mold 13 is formed of a material having a gas permeability, the water-soluble polymer-dissolved solution 22 containing a medicament can be suctioned by suctioning the solution from the rear surface of the mold 13, and the filling of the needle-like recesses 15 with the water-soluble polymer-dissolved solution 22 containing a medicament can be promoted.

Next to the filling process of FIG. 13B, the lip portion 34A of the nozzle 34 is brought into contact with the surface of the mold 13, the liquid supply device 36 is relatively moved in the length direction and the vertical direction of the opening portion 34B, and the nozzle 34 is moved to the needle-like recesses 15 which are not filled with the water-soluble polymer-dissolved solution 22 containing a medicament. The position of the opening portion 34B of the nozzle 34 is adjusted on the needle-like recesses 15, as illustrated in FIG. 13C. In the present embodiment, the example of moving the nozzle 34 has been described, but the mold 13 may be moved.

Since the lip portion 34A of the nozzle 34 is brought into contact with the surface of the mold 13 and then the movement is made, the water-soluble polymer-dissolved solution 22 containing a medicament, which remains on the surface other than the needle-like recesses 15 of the mold 13 can be collected by the nozzle 34. It is possible to prevent the water-soluble polymer-dissolved solution 22 containing a medicament from remaining on the surface other than the needle-like recesses 15 of the mold 13.

In order to reduce the damage to the mold 13 and suppress deformation due to compression of the mold 13 as much as possible, it is preferable that the pressing pressure of the nozzle 34 against the mold 13 is set to be as small as possible during the movement. Further, in order to prevent the water-soluble polymer-dissolved solution 22 containing a medicament from remaining on the surface other than the needle-like recesses 15 of the mold 13, it is preferable that at least one of the mold 13 or the nozzle 34 is formed of a flexible material which can be elastically deformed.

By repeating the filling process of FIG. 13B and the moving process of FIG. 13C, 5 rows and 5 columns of needle-like recesses 15 which are two dimensionally arranged are filled with the water-soluble polymer-dissolved solution 22 containing a medicament. When 5 rows and 5 columns of needle-like recesses 15 which are two dimensionally arranged are filled with the water-soluble polymer-dissolved solution 22 containing a medicament, the liquid supply device 36 is moved to 5 rows and 5 columns of two-dimensionally arranged needle-like recesses 15 which are adjacent to the needle-like recesses filled with the solution and then the filling process of FIG. 13B and the moving process of FIG. 13C are repeated. The 5 rows and 5 columns of two-dimensionally arranged needle-like recesses 15 which are adjacent to the needle-like recesses filled with the solution are filled with the water-soluble polymer-dissolved solution 22 containing a medicament.

For the above-described filling process and moving process, (1) embodiment in which the needle-like recesses 15 are filled with the water-soluble polymer-dissolved solution 22 containing a medicament while the nozzle 34 is moved or (2) embodiment in which the nozzle 34 is temporarily stopped on the needle-like recesses 15 during the movement of the nozzle 34, the needle-like recesses 15 are filled with the water-soluble polymer-dissolved solution 22 containing a medicament, and the nozzle 34 is moved again after the filling may be adopted. The lip portion 34A of the nozzle 34 is brought into the surface of the mold 13 between the filling process and the moving process.

FIG. 15 is a partially enlarged view of the mold 13 and the tip of the nozzle 34 at the time of filling the needle-like recess 15 with the water-soluble polymer-dissolved solution 22 containing a medicament. As illustrated in FIG. 15, the filling of the needle-like recess 15 with the water-soluble polymer-dissolved solution 22 containing a medicament can be promoted by applying a pressing force P1 into the nozzle 34. Further, when the needle-like recess 15 is filled with the water-soluble polymer-dissolved solution 22 containing a medicament, it is preferable that a pressing pressure P2 for bringing the nozzle 34 into contact with the surface of the mold 13 is set to be greater than or equal to the pressing force P1 applied into the nozzle 34. When the pressing pressure P2 is set to be greater than or equal to the pressing force P1, it is possible to suppress leaking of the water-soluble polymer-dissolved solution 22 containing a medicament to the surface of the mold 13 from the needle-like recess 15.

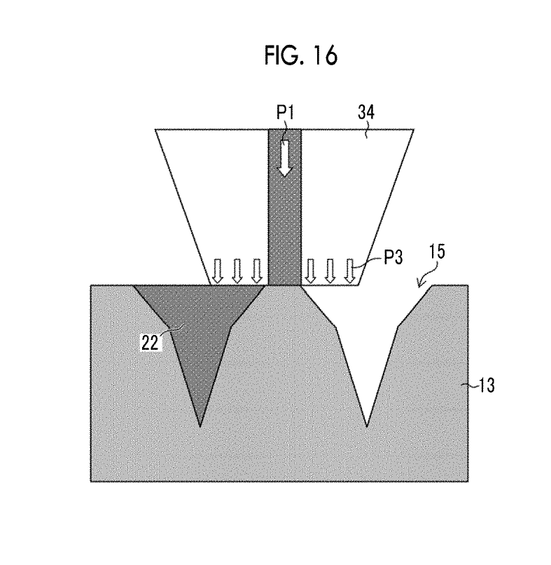

FIG. 16 is a partially enlarged view of the tip of the nozzle 34 and the mold 13 during the movement of the nozzle 34. When the nozzle 34 is relatively moved with respect to the mold 13, it is preferable that a pressing pressure P3 of bringing the nozzle 34 into contact with the surface of the mold 13 is set to be smaller than the pressing pressure P2 of bringing the nozzle 34 into contact with the surface of the mold 13 during the filling. When the pressing pressure P3 is set to be smaller than the pressing force P2, the damage to the mold 13 is reduced and the deformation of the mold 13 due to compression is suppressed.

When the filling of the plurality of needle-like recesses 15 formed of 5 rows and 5 columns of needle-like recesses is completed, the nozzle 34 is moved to the plurality of needle-like recesses 15 formed of 5 rows and 5 columns of needle-like recesses adjacent to the needle-like recesses filled with the solution. When the nozzle 34 is moved to the plurality of needle-like recesses 15 formed of 5 rows and 5 columns of needle-like recesses adjacent to the needle-like recesses filled with the solution at the time of liquid supply, it is preferable that the supply of the water-soluble polymer-dissolved solution 22 containing a medicament is stopped. There is a distance between the needle-like recesses 15 in the fifth row and the needle-like recesses 15 in the next first row. When the water-soluble polymer-dissolved solution 22 containing a medicament is continuously supplied during the movement of the nozzle 34 between the rows, the liquid pressure inside of the nozzle 34 is extremely high in some cases. As the result, the water-soluble polymer-dissolved solution 22 containing a medicament supplied from the nozzle 34 occasionally flows out of the needle-like recesses 15 of the mold 13. In order to prevent the solution from flowing out, it is preferable that the liquid pressure inside the nozzle 34 is detected and the supply of the water-soluble polymer-dissolved solution 22 containing a medicament is stopped when it is determined that the liquid pressure is extremely high.

In the above, the method of supplying the water-soluble polymer-dissolved solution containing a medicament using a dispenser that has a nozzle has been described, but bar coating, spin coating, or spray coating can be applied in addition to the coating with the dispenser.

In the present invention, it is preferable that the drying treatment is performed after the water-soluble polymer-dissolved solution containing a medicament is supplied to the needle-like recesses.

Preferably, the microneedle array of the present invention can be produced by performing a process of forming some needles by drying a mold for forming needles, filled with the first water-soluble polymer-dissolved solution containing a medicament; and a process of filling the upper surface of some needles formed in the above-described manner with the second water-soluble polymer-dissolved solution and drying the mold.

It is preferable that the mold for forming needles, filled with the first water-soluble polymer-dissolved solution containing a medicament is dried under the condition in which the moisture content of the first water-soluble polymer-dissolved solution reaches 20% or less after 300 minutes from when 30 minutes elapse after the drying is started.

It is particularly preferable that the drying is controlled such that the temperature is maintained at which the medicament does not lose its effect and the moisture content of the first water-soluble polymer-dissolved solution reaches 20% or less after at least 60 minutes elapse from when the drying is started.

As a method of controlling the above-described drying speed, arbitrary means capable of delaying the drying, for example, the temperature, the humidity, the drying air volume, the use of a container, or the volume and/or the shape of the container can be selected.

Preferably, the mold for forming needles, filled with the first water-soluble polymer-dissolved solution containing a medicament can be dried in a state in which the mold is covered by a container or the mold is accommodated in a container.

The temperature of drying is preferably in a range of 1° C. to 45° C. and more preferably in a range of 1° C. to 40° C.

The relative humidity of drying is preferably in a range of 10% to 95%, more preferably in a range of 20% to 95%, and still more preferably in a range of 30% to 95%.

(Formation of Sheet)

Several embodiments of a process of forming the sheet will be described.

A first embodiment of a process of forming the sheet will be described with reference to FIGS. 17A to 17D. The needle-like recesses 15 of the mold 13 are filled with the water-soluble polymer-dissolved solution 22 containing a medicament from the nozzle 34. Next, a layer 120 containing a medicament in the needle-like recesses 15 is formed by drying and solidifying the water-soluble polymer-dissolved solution 22 containing a medicament as illustrated in FIG. 17B. Subsequently, the mold 13 on which the layer 120 containing a medicament is formed is coated with the water-soluble polymer-dissolved solution 24 using a dispenser as illustrated in FIG. 17C. In addition to the coating with the dispenser, bar coating, spin coating, or spray coating can be applied. Since the layer 120 containing a medicament is solidified, it is possible to prevent the medicament from being diffused in the water-soluble polymer-dissolved solution 24. Next, the microneedle array 1 including a plurality of needles 112, frustums 113, and the sheet 116 is formed by drying and solidifying the water-soluble polymer-dissolved solution 24 as illustrated in FIG. 17D.

In the first embodiment, in order to promote the filling of the needle-like recesses 15 with the water-soluble polymer-dissolved solution 22 and the water-soluble polymer-dissolved solution 24 containing a medicament, it is preferable to apply a pressure from the surface of the mold 13 and perform suctioning from the rear surface of the mold 13 under reduced pressure.

Subsequently, a second embodiment of a process will be described with reference to FIGS. 18A to 18C. The needle-like recesses 15 of the mold 13 are filled with the water-soluble polymer-dissolved solution 22 containing a medicament from the nozzle 34 as illustrated in FIG. 18A. Next, similar to FIG. 17B, the layer 120 containing a medicament is formed in the needle-like recesses 15 by drying and solidifying the water-soluble polymer-dissolved solution 22 containing a medicament. Next, another support 29 is coated with the water-soluble polymer-dissolved solution 24 as illustrated in FIG. 18B. The support 29 is not limited, and examples of the support include polyethylene, polyethylene terephthalate, polycarbonate, polypropylene, an acrylic resin, triacetyl cellulose, and glass. Subsequently, the water-soluble polymer-dissolved solution 24 formed on the support 29 overlaps with the mold 13 having the layer 120 containing a medicament formed on the needle-like recesses 15 as illustrated in FIG. 18C. In this manner, the needle-like recesses 15 are filled with the water-soluble polymer-dissolved solution 24. Since the layer containing a medicament is solidified, it is possible to prevent the medicament from being diffused in the water-soluble polymer-dissolved solution 24. Next, the microneedle array including a plurality of needles 112, frustums 113, and the sheet 116 is formed by drying and solidifying the water-soluble polymer-dissolved solution 24.

In the second embodiment, in order to promote the filling of the needle-like recesses 15 with the water-soluble polymer-dissolved solution 24, it is preferable to apply a pressure from the surface of the mold 13 and perform decompressing and suctioning from the rear surface of the mold 13.

As the method of drying the water-soluble polymer-dissolved solution 24, a process of volatilizing the solvent in the polymer-dissolved solution may be exemplified. The method is not particularly limited, and a method of performing heating, blowing air, or decompression may be used. The drying treatment can be performed under the conditions of 1° C. to 50° C. for 1 to 72 hours. Examples of the method of blowing air include a method of blowing hot air at 0.1 to 10 m/sec. It is preferable that the drying temperature is set to a temperature at which the medicament in the polymer-dissolved solution 22 containing a medicament is not thermally deteriorated.

(Peeling)

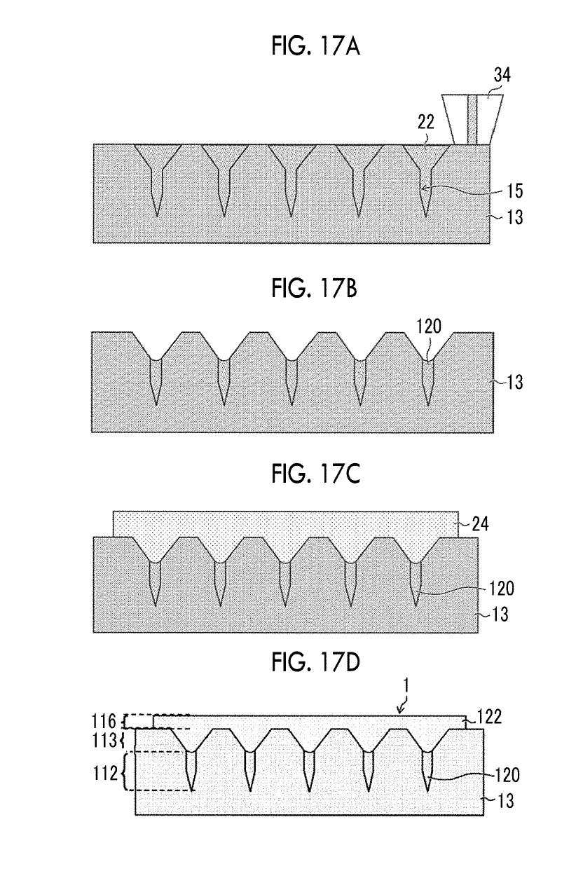

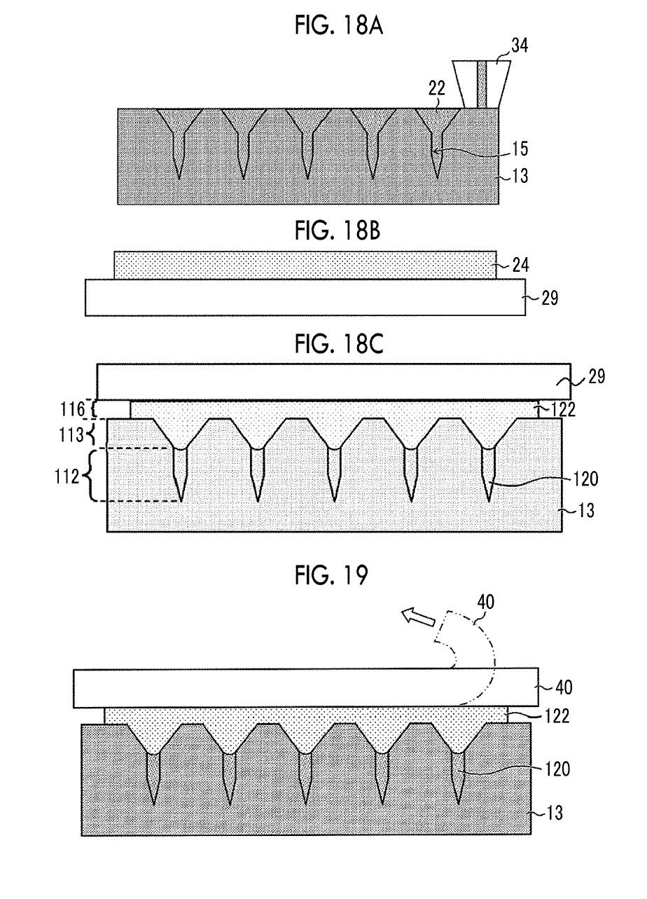

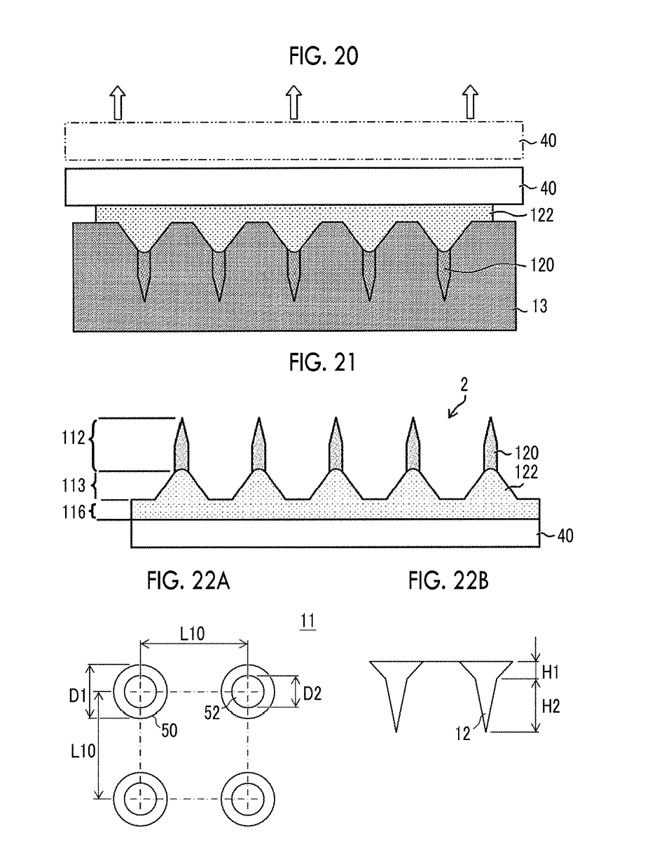

A method of peeling the microneedle array from the mold 13 is not particularly limited. It is preferable that needles are not bent or broken at the time of peeling. Specifically, a sheet-like base material 40 on which a pressure sensitive adhesive layer is formed is attached to the microneedle array and then the base material 40 can be peeled off from the end portion such that the base material 40 is turned over as illustrated in FIG. 19. However, the needles can be bent when this method is used. Therefore, as illustrated in FIG. 20, a sucking disc (not illustrated) is disposed on the base material 40 on the microneedle array so that a method of vertically pulling the base material up while suctioning the base material with air can be applied. Further, the support 29 may be used as the base material 40.

FIG. 21 illustrates the microneedle array 2 peeled from the mold 13. The microneedle array 2 includes the base material 40, the needles 112 formed on the base material 40, the frustums 113, and the sheet 116. At least the tip of the needle 112 has a conical shape or a polygonal pyramid shape, but the shape of the needle 112 is not limited thereto.

The method of producing the microneedle array of the present invention is not particularly limited, but it is preferable that the microneedle array is obtained by a production method including (1) a process of producing a mold; (2) a process of preparing a medicament and a water-soluble polymer; (3) a process of filling the mold with the liquid obtained in the process (2) and forming a needle tip region; (4) a process of filling the mold with the water-soluble polymer and forming remaining needles (frustums if desired) and the sheet; and (5) a process of peeling the microneedle array from the mold.

The microneedle array of the present invention can be administered to a subject by puncturing a surface of skin of the subject with the microneedle array. The subject may include any mammals such as human. The subject is preferably human. The administration period (time for puncture) is not particularly limited, and is generally from 1 second to 1 hour, and is preferably from 1 minute to 30 minutes, and is more preferably from 1 minute to 20 minutes.

Hereinafter, the present invention will be described in detail with reference to examples. The materials, the amounts to be used, the ratios, the treatment contents, and the treatment procedures shown in the examples described below can be appropriately changed as long as they are within the gist of the present invention. Accordingly, the scope of the present invention should not be limitatively interpreted by the specific examples described below.

### EXAMPLES

<Preparation of Human Growth Hormone (hGH)-Containing Microneedle Array A Having L of 600 μm and L1 of 400 μm>

(Production of Mold)

An original plate 11 was prepared by arranging shaped portions 12 having a needle-like structure, on which a cone 52 with a diameter D2 of 300 μm and a height H2 of 600 μm was formed, as illustrated in FIG. 22, on a truncated cone 50 having a bottom surface with a diameter D1 of 500 μm and having a height H1 of 150 μm on the surface of a smooth Ni plate in which each side had a length of 40 mm and performing grinding processing on 100 needles having a pitch L10 of 1000 μm and a square pyramid shape in a two-dimensional square array. The original plate 11 was covered by silicon rubber (SILASTIC MDX 4-4210, manufactured by Dow Corning Toray Co., Ltd.) to form a film having a thickness of 0.6 mm and the film was thermally cured in a state in which 50 μm of the conical tip of the original plate 11 protruded from the film surface and then peeled off. In this manner, an inverted product of the silicon rubber having a through-hole with a diameter of approximately 30 μm was prepared. The silicon rubber inverted product which had 10 rows and 10 columns of needle-like recesses two-dimensionally arranged being formed on the central portion and in which the portion other than the flat surface portion in which each side had a length of 30 mm was cut off was used as a mold. A surface on which the opening portion of a needle-like recess was wide was set to the surface of the mold and a surface having a through-hold (air vent hole) with a diameter of 30 μm was set to the rear surface of the mold.

(Preparation of Water-Soluble Polymer-Dissolved Solution Containing Human Growth Hormone as Medicament)

Human growth hormone (hGH) (GENOTROPIN (registered trademark), Pfizer Inc.) was concentrated by a centrifugal ultrafiltration method and mixed with hydroxyethyl starch (HES) (Fresenius Kabi) and sucrose (Suc) (Japanese Pharmacopoeia grade, Wako Pure Chemical Industries, Ltd.), and an aqueous solution in which the amount of hGH was 50 mg/mL, the amount of HES was 25 mg/mL, and the amount of Suc was 25 mg/mL was prepared.

(Preparation of Water-Soluble Polymer-Dissolved Solution Forming Sheet)

Chondroitin sulfate (manufactured by Maruha Nichiro Corporation) was dissolved in water such that the content thereof was set to 40% by mass, and a water-soluble polymer-dissolved solution forming a sheet and a region lower than the length L1 of a needle was prepared.

(Filling and Drying of Polymer-Dissolved Solution Containing Human Growth Hormone)

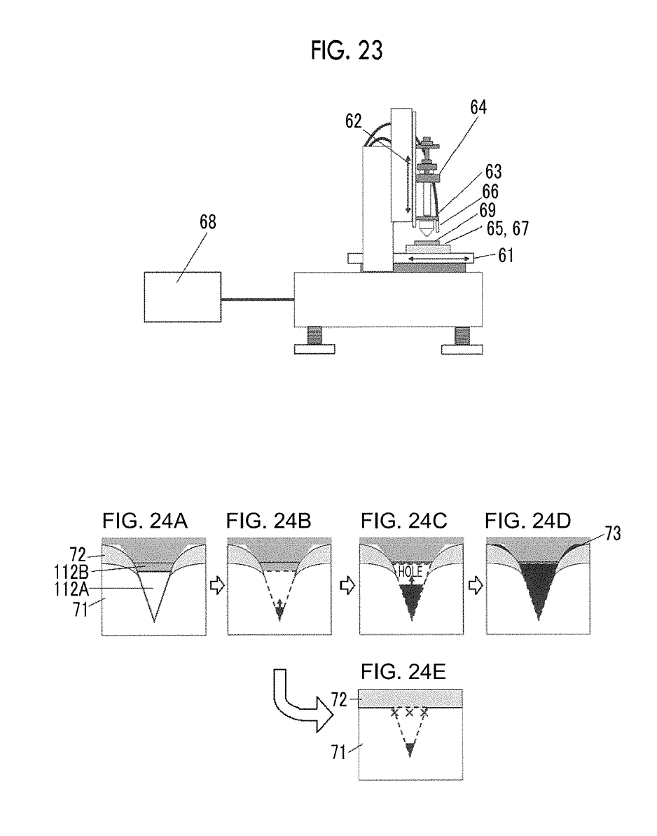

A filling device illustrated in FIG. 23 was used. The filling device includes an X-axis drive unit 61 and a Z-axis drive unit 62 which control relative position coordinates of the mold and the nozzle; a liquid supply device 64 (ultratrace determination dispenser SMP-III, manufactured by Musashi Engineering, Inc.) to which the nozzle 63 is attachable; a suction stand 65 which fixes the mold 69; a laser displacement meter 66 (HL-C201A, manufactured by Panasonic Corporation) which measures the shape of the mold surface; a load cell 67 (LCX-A-500N, manufactured by Kyowa Electronic Instruments Co., Ltd.) which measures the pressing pressure of the nozzle; and a control mechanism 68 which controls the Z-axis based on data of measured values of the surface shape and the pressing pressure.

An air permeating film (Poreflon (registered trade mart), FP-010, manufactured by Sumitomo Electric Industries, Ltd.) in which each side had a length of 15 mm was placed on a horizontal suction stand, and the mold was disposed on the surface thereof such that the surface of the mold faced up. The air permeating film and the mold were fixed to a vacuum stand by performing decompression in the rear surface direction of the mold with a suction pressure of a gauge pressure of 90 kPa.

A stainless steel (SUS) nozzle in a shape illustrated in FIG. 14 was prepared, and a slit-like opening portion having a length of 12 mm and a width of 0.2 mm was formed in the center of a lip portion having a length of 20 mm and a width of 2 mm. This nozzle was connected to a liquid supply device. The liquid supply device and the inside of the nozzle were filled with a 3 mL water-soluble polymer-dissolved solution containing a medicament. The nozzle was adjusted such that the opening portion was set to be in parallel with a plurality of needle-like recesses in the first row, formed on the surface of the nozzle. The nozzle was pressed to the mold at a pressure of 1.372×10⁴ Pa (0.14 kgf/cm²) in a position spaced by 2 mm in a direction opposite to the second row with respect to the first row. The water-soluble polymer-dissolved solution containing a medicament was allowed to be released from the opening portion at 0.15 μL/sec for 20 seconds in the liquid supply device while the nozzle was moved in the length direction and the vertical direction of the opening portion at 0.5 mm/sec while the nozzle was pressed and the Z axis was controlled such that the fluctuation in the pressing pressure was in a range of ±0.490×104 Pa (0.05 kgf/cm²). The movement of the nozzle was stopped in a position spaced by 2 mm after the nozzle passed through the hole pattern of the plurality of needle-like recesses two-dimensionally arranged and then the nozzle was peeled from the mold.

The mold filled with the water-soluble polymer-dissolved solution containing a medicament was stored in a boxy in an environment of a temperature of 23° C. and a relative humidity of 45% and then dried. At this time, the water-soluble polymer-dissolved solution containing a medicament was gradually dried and the moisture content thereof became 20% or less after 60 minutes elapsed. Further, the means for drying is not limited to a lid and other means such as control of the temperature and the humidity or control of the air volume may be used.

(Forming and Drying Sheet)

As a support of forming the sheet, a support on which a hydrophilized plasma treatment was performed under the following conditions (used gas: O2, gas pressure: 13 Pa, high frequency (RF) powder: 100 W, irradiation time: 3 minutes, O2 flow rate: SV250, target vacuum degree (CCG): 2.0×10⁻⁴ Pa) using a polyethylene terephthalate (PET) sheet (175 μm) and a cloud remover (Victor Jvc, Ltd.) was used. The PET subjected to the treatment was coated with the water-soluble polymer-dissolved solution such that the front and rear surfaces had a film thickness of 75 μm. Further, the mold filled with the water-soluble polymer-dissolved solution containing a medicament was suctioned and fixed to the suction stand. The surface of the PET coated with the water-soluble polymer-dissolved solution was disposed to face the surface of the mold and the interval between the PET and the mold and the interval between the PET and the space on a side opposite to the mold were decompressed for 2 minutes. After the decompression, the PET coated with the water-soluble polymer-dissolved solution and the mold were bonded to each other by releasing the atmospheric pressure only in the interval between the PET and the space on a side opposite to the mold. The resultant formed by the PET and the mold being bonded to each other and being integrated with each other was dried after the state in which the PET was in contact with the mold was maintained for 10 minutes.

(Peeling)

The dried and solidified microneedle array was carefully peeled off from the mold, thereby forming a microneedle array containing human growth hormone. The microneedle array includes a sheet, frustums, and needles. The length L of a needle is approximately 600 μm and the width of a base portion of a needle is approximately 270 μm. The frustum has a truncated cone structure, the height of the frustum is approximately 130 μm, the diameter of the upper bottom surface of the frustum is approximately 270 μm, and the diameter of the lower bottom surface of the frustum is approximately 500 μm. The thickness of the sheet is approximately 205 μm (the thickness of polyethylene terephthalate is approximately 175 μm). The number of needles is 100, the interval between needles is approximately 1 mm, and the needles are arranged in the square form.

(Distribution of Human Growth Hormone in Needles of Human Growth Hormone (hGH)-Containing Microneedle Array A)

A part at a height of 400 μm from the tips of needles of the prepared microneedle array A was cut in parallel with the sheet. The cut needles, a part at a height lower than 400 μm from the tips of the needles of the microneedle array, and the sheet were respectively immersed in water and dissolved therein for 0.5 hours. The amount of human growth hormone in each solution was measured by a size exclusion chromatography method, and it was confirmed that a microneedle array containing 90% of the total medicament in the microneedle array at a height of 400 μm from the tips of needles was obtained.

(Test for Confirming Average Remaining Length L2 of Needle after Mini Pig was Punctured by Human Growth Hormone (hGH) Containing Microneedle Array A)

The hair on the back of a mini pig (Gottingen mini-pig, male, 5 weeks old, approximately 10 kg) was removed under anesthesia, the back was punctured by the human growth hormone (hGH)-containing microneedle array A, and the microneedle array A was peeled off from the back after the time listed in Table 1 elapsed.

The back was punctured by the microneedle array A at different time and the remaining length of needles of the microneedle array after the puncture was measured using a stereoscopic microscope. The measurement was performed on 100 needles in total and the average value was set to the remaining length L2. The measurement results are listed in Table 1.

TABLE 1

| | Time for | Remaining length |
|---|---|---|
| | puncture | (L2) |
| Example 1 | 10 minutes | 34 μm |
| Example 2 | 10 minutes | 54 μm |
| Comparative | 5 minutes | 330 μm |
| Example 1 | | |

It was confirmed that the remaining length L2 was able to be shortened by increasing the time for puncture as listed in Table 1.

(Blood Kinetics Test of Human Growth Hormone (hGH)-Containing Microneedle Array A in Mini Pig)

The hair on the back of a mini pig (Gottingen mini-pig, male, 5 weeks old, approximately 10 kg) was removed under anesthesia, the back was punctured by the human growth hormone (hGH)-containing microneedle array A, and the microneedle array A was peeled off from the back after 10 minutes elapsed. In order to evaluate relative bioavailability with respect to subcutaneous injection (S. C), the same amount of hGH as in target microneedles was subcutaneously injected to the mini pig under the same conditions as described above.

Blood sampling over time was performed by collecting 0.5 mL of blood each time through a catheter at each time of 5 minutes, 15 minutes, 20 minutes, 30 minutes, 45 minutes, 1 hour, 2 hours, 4 hours, and 6 hours before and after the administration. The plasma solution was recovered from the collected blood and the amount of hGH in the plasma was measured according to an enzyme immunosorbent assay (ELISA) method. The amount of hGH in the blood was graphed, the area under the blood concentration-time curve (AUC) was calculated, and then relative bioavailability with respect to subcutaneous injection was calculated. When the relative bioavailability with respect to subcutaneous injection was 70% or greater, this was evaluated as A. Further, when the relative bioavailability with respect to subcutaneous injection was less than 70%, this was evaluated as B. The measurement results of the relative bioavailability are listed in Table 2.

(Measurement of Remaining Length L2 of Microneedle Array after Administration)

The remaining length of needles of the microneedle array after being administered to a mini pig was measured using a stereoscopic microscope. The measurement was performed on 100 needles in total and the average value was set to the remaining length L2. The measurement results of L2 are listed in Table 2.

TABLE 2

| | Remaining | Relative | Evaluation of relative |
|---|---|---|---|
| | length (L2) | bioavailability | availability |
| Comparative | 260 μm | 69% | B |
| Example 2 | | | |
| Example 3 | 73 μm | 125% | A |
| Example 4 | 81 μm | 79% | A |
| Example 5 | 106 μm | 71% | A |
| Example 6 | 59 μm | 82% | A |
| Example 7 | 87 μm | 79% | A |
| Example 8 | 35 μm | 78% | A |
| Example 9 | 66 μm | 95% | A |
| Example 10 | 54 μm | 78% | A |

From the results listed in Table 2, it was understood that each group of examples in which the remaining length L2 was greater than or equal to 20 μm and less than or equal to 200 μm (L-L1) had excellent drug efficacy compared to a group of the comparative example in which L2 was less than 200 μm.

The above-described test results show that most of the medicament in the microneedle array was transferred into the blood and the medicament administered through holes in the skin which were opened by the needles of the microneedle array was able to be administered without flowing out of the skin by administering the microneedle array such that a region containing a medicament in needles of the microneedle array was completely dissolved and the root region which did not contain a medicament in needles or had a small content of the medicament was not dissolved, and thus the same drug efficacy as in the case of subcutaneous injection was able to be obtained.

<Preparation of Human Growth Hormone (hGH)-Containing Microneedle Array B Having L of 600 μm and L1 of 500 μm>

(Production of Mold)

A human growth hormone (hGH)-containing microneedle array B was prepared using the same method as in the case of the human growth hormone (hGH)-containing microneedle array A.

(Preparation of Water-Soluble Polymer-Dissolved Solution Containing Human Growth Hormone)

Human growth hormone (hGH) (GENOTROPIN (registered trademark), Pfizer Inc.) was concentrated by a centrifugal ultrafiltration method and mixed with hydroxyethyl starch (HES) (Fresenius Kabi) and sucrose (Suc) (Japanese Pharmacopoeia grade, Wako Pure Chemical Industries, Ltd.), and an aqueous solution in which the amount of hGH was 50 mg/mL, the amount of HES was 100 mg/mL, and the amount of Suc was 100 mg/mL was prepared.

(Preparation of Water-Soluble Polymer-Dissolved Solution Forming Sheet)

A water-soluble polymer-dissolved solution was prepared using the same method as in the case of the human growth hormone (hGH)-containing microneedle array A.

(Filling and Drying of Polymer-Dissolved Solution Containing Human Growth Hormone)

Filling and drying of the polymer-dissolved solution containing human growth hormone were performed as in the case of the human growth hormone (hGH)-containing microneedle array A.

(Forming and Drying Sheet)

Forming and drying the sheet were performed as in the case of the human growth hormone (hGH)-containing microneedle array A.

(Peeling)

Peeling was performed as in the case of the human growth hormone (hGH)-containing microneedle array A.

(Distribution of Human Growth Hormone in Needles of Human Growth Hormone (hGH)-Containing Microneedle Array B)

A part at a height of 500 μm from the tips of needles of the prepared microneedle array B was cut in parallel with the sheet. The cut needles, a part at a height lower than 500 μm from the tips of the needles of the microneedle array, and the sheet were respectively immersed in water and dissolved therein for 0.5 hours. The amount of human growth hormone in each solution was measured by a size exclusion chromatography method, and it was confirmed that a microneedle array containing 90% of the total medicament in the microneedle array at a height of 500 μm from the tips of needles was obtained.

(Test for Confirming Average Remaining Length L2 of Needle after Mini Pig was Punctured by Human Growth Hormone (hGH) Containing Microneedle Array B)

The hair on the back of a mini pig (Gottingen mini-pig, male, 5 weeks old, approximately 10 kg) was removed under anesthesia, the back was punctured by the human growth hormone (hGH)-containing microneedle array B, and the microneedle array B was peeled off from the back after the time listed in Table 3 elapsed.

The remaining length L2 of the peeled microneedle array was measured using the same method as in the case of the human growth hormone (hGH)-containing microneedle array A. The measurement results are listed in Table 3.

TABLE 3

| | Time for | Remaining |
|---|---|---|
| | puncture | length (L2) |
| Example 11 | 10 minutes | 38 μm |
| Example 12 | 10 minutes | 60 μm |
| Comparative | 5 minutes | 153 μm |
| Example 3 | | |
| Comparative | 30 minutes | 0 μm |
| Example 4 | | |

It was confirmed that the remaining length L2 was able to be shortened by increasing the time for puncture as listed in Table 3.

(Blood Kinetics Test of Human Growth Hormone (hGH)-Containing Microneedle Array B in Mini Pig)

The blood kinetics test of the human growth hormone (hGH)-containing microneedle array B in a mini pig was performed using the same method as in the case of the human growth hormone (hGH)-containing microneedle array A. The evaluation results of relative bioavailability were listed in Table 4.

(Measurement of Remaining Length L2 of Microneedle Array After Administration)

The measurement of the remaining length L2 of the microneedle array was performed using the same method as in the case of the human growth hormone (hGH)-containing microneedle array A. The measurement results of L2 are listed in Table 4.

TABLE 4

| | Remaining length | Relative | Evaluation of relative |
|---|---|---|---|
| | (L2) | bioavailability | bioavailability |
| Comparative | 116 μm | 40% | B |
| Example 5 | | | |
| Comparative | 18 μm | 46% | B |
| Example 6 | | | |
| Comparative | 16 μm | 40% | B |
| Example 7 | | | |
| Example 13 | 89 μm | 93% | A |
| Example 14 | 61 μm | 140% | A |
| Example 15 | 49 μm | 103% | A |
| Example 16 | 58 μm | 73% | A |

From the results listed in Table 4, it was understood that each group of examples in which the remaining length L2 was greater than or equal to 20 μm and less than or equal to 100 μm (L-L1) had excellent drug efficacy compared to a group of the comparative example in which L2 was less than 20 μm or L2 was greater than 100 μm.

The above-described test results show that most of the medicament in the microneedle array was transferred into the blood and the medicament administered through holes in the skin which were opened by the needles of the microneedle array was able to be administered without flowing out of the skin by administering the microneedle array such that a region containing a medicament in needles of the microneedle array was completely dissolved and the root region which did not contain a medicament in needles or had a small content of the medicament was not dissolved, and thus the same drug efficacy as in the case of subcutaneous injection was able to be obtained.

Further, the leaked-out medicaments of Comparative Example 7 and Example 13 were quantified. A phosphate buffer solution was added dropwise to the surface of the skin after the microneedle array was peeled off, the same solution was recovered, and the contained medicament was quantified. As the result, it was confirmed that 35% of the amount of medicament contained in the microneedle array was contained in the solution in Comparative Example 7 and 12% of the amount of medicament contained in the microneedle array was contained in the solution in Example 13. In this manner, in Example 13 in which the remaining length L2 was greater than and equal to 20 μm and less than or equal to 100 μm (L−L1), the leaking out of the medicament was suppressed.

### EXPLANATION OF REFERENCES

1: microneedle array

2: microneedle array

110: microneedle

112: needle

112A: needle tip region

112B: needle root region

113: frustum

116: sheet

120: layer containing medicament

122: layer which does not contain medicament

L:length of needle

L1: length of needle tip region from needle tip

W: diameter (width)

H: height

T: height (thickness)

11: original plate

12: shaped portion

13: mold

15: needle-like recess

15A: inlet

15B: tip recess

15C: air vent hole

D: diameter (diameter)

18: mold complex

19: air permeating sheet

20: base

22: water-soluble polymer-dissolved solution containing medicament

24: water-soluble polymer-dissolved solution

29: support

30: tank

32: pipe

34: nozzle

34A: lip portion

34B: opening portion

36: liquid supply device

P1: pressing force

P2: pressing pressure

P3: pressing pressure

40: base material

50: truncated cone

52: cone

D1: diameter

D2: diameter

L10: pitch

H1: height

H2: height

61: X-axis drive unit

62: Z-axis drive unit

63: nozzle

64: liquid supply device

65: suction stand

66: laser displacement meter

67: load cell

68: control mechanism

69: mold

71: hypodermis

72: epidermis

73: leaked-out medicament

What is claimed is:

1. A microneedle array comprising:
a sheet; and
a plurality of needles present on an upper surface of the sheet,
wherein the needles contain a water-soluble polymer and a medicament,
the sheet contains a water-soluble polymer, and
administration is performed such that 20 μm≦L2≦L−L1 is satisfied,
where, L represents a length of a needle,
L1 represents a length of a needle tip region, which contains 90% of the total medicament in the microneedle array, from a needle tip,
L2 represents an average remaining length of the needle after the administration using the microneedle array, and
a unit of L, L1, and L2 is μm.

2. The microneedle array according to claim 1,
wherein the administration is performed such that 20 μm≦L2=L−L1 is satisfied.

3. The microneedle array according to claim 1, further comprising:
a plurality of frustums which are present between the sheet and the plurality of needles,
wherein the needle tip region contains a first water-soluble polymer and a medicament, and
a root region other than the needle tip region, a frustum, and the sheet of the needle contain a second water-soluble polymer.

4. The microneedle array according to claim 1,
wherein the needle tip region further contains disaccharides.

5. The microneedle array according to claim 4,
wherein the disaccharides are one or more selected from sucrose, maltose, and trehalose.

6. The microneedle array according to claim 1,
wherein the water-soluble polymers of a needle and a sheet each independently are at least one selected from the group consisting of hydroxyethyl starch, dextran, chondroitin sulfate, sodium chondroitin sulfate, sodium hyaluronate, carboxymethyl cellulose, polyvinylpyrrolidone, polyoxyethylene polyoxypropylene glycol, polyethylene glycol, and polyvinyl alcohol.

7. The microneedle array according to claim 1,
wherein the medicament is a peptide hormone.

8. A method of administering a microneedle array comprising: administering a microneedle array which includes a sheet; and a plurality of needles present on an upper surface of the sheet and in which the needles contain a water-soluble polymer and a medicament, and the sheet contains a water-soluble polymer, to a subject such that 20 μm≦L2≦L−L1 is satisfied, where, L represents a length of a needle, L1 represents a length of a needle tip region, which contains 90% of the total medicament in the microneedle array, from a needle tip, L2 represents an average remaining length of needles after administration using the microneedle array, and a unit of L, L1, and L2 is μm.

9. The method of administering a microneedle array according to claim 8, in which the administration is performed such that 20 μm≦L2=L−L1 is satisfied.

10. The method of administering a microneedle array according to claim 8, in which the microneedle array includes a plurality of frustums between the sheet and the plurality of needles, the needle tip region contains a first water-soluble polymer and a medicament, and a root region other than the needle tip region, a frustum, and the sheet of the needle contain a second water-soluble polymer.

11. The method of administering a microneedle array according to claim 8, in which the needle tip region further contains disaccharides.

12. The method of administering a microneedle array according to claim 11, in which the disaccharides are one or more selected from sucrose, maltose, and trehalose.

13. The method of administering a microneedle array according to claim 8, in which the water-soluble polymers of a needle and a sheet each independently are at least one selected from the group consisting of hydroxyethyl starch, dextran, chondroitin sulfate, sodium chondroitin sulfate, sodium hyaluronate, carboxymethyl cellulose, polyvinylpyrrolidone, polyoxyethylene polyoxypropylene glycol, polyethylene glycol, and polyvinyl alcohol.

14. The method of administering a microneedle array according to claim 8, in which the medicament is a peptide hormone.
